(19) United States
(12) Patent Application Publication
(10) Pub. No.: US 2011/0108024 A1
(43) Pub. Date: May 12, 2011

(54) Timer for nebulizer compressor

(76) Inventor: Jesus N. Padilla, Rialto, CA (US)

(21) Appl. No.: 12/925,905

(22) Filed: Nov. 2, 2010

Related U.S. Application Data
(60) Provisional application No. 61/281,081, filed on Nov. 12, 2009.

(51) Int. Cl. A61M 16/00

(52) U.S. Cl. 128/200.14

(57) ABSTRACT

The Timer For Nebulizer Compressor provides a shatterproof plastic casing which houses the unit motor and a piston driven or comparable compressor. Associated accessories such as a mouthpiece, medicine cup and elongated, flexible tubing are also included. The Timer For Nebulizer Compressor runs on standard AC power and can be connected to any power outlet. The novel feature of the Timer For Nebulizer Compressor however, is found in the previously mentioned timer. This timer is directly connected to the unit's shut off valve. As such, a therapist simply prepares the unit for use, at which point the timer is set for the time period required to administer the designated course of treatment. When the treatment was complete and the allotted time had passed, the timer is activated, automatically shutting the nebulizer off and thus preventing the unit from overheating.

### CLAIM OF PRIORITY

This patent application claims priority under 35 USC 119 (e) (1) from U.S. Provisional Patent Application Ser. No. 61/281,081 filed Nov. 12, 2009, of common inventorship herewith entitled, “Timer for nebulizer compressor.”

### FIELD OF THE INVENTION

The present invention pertains to the field of accessories of nebulizers and more specifically to the field of timers for nebulizers.

### BACKGROUND OF THE INVENTION

The prior art has put forth several designs for timers for nebulizers. Among these are:

U.S. Pat. No. 4,624,251 to Nicholas C. Miller describes and apparatus for administering a nebulized substance. The nebulizer described herein comprises a nebulizer and a timer for determining the period of time during which aerosol is permitted to pass through the delivery means.

U.S. Pat. No. 6,732,731 to Daniel C. M. Tseng describes a medical mechanical compressor nebulizer.

U.S. Pat. No. 7,267,121 to Ehud Ivri describes an aerosol delivery apparatus and method for pressure-assisted breathing systems.

None of these prior art references describe the present invention.

### SUMMARY OF THE INVENTION

It is an object of the present invention to provide an improved nebulizer for the aerosolization of pharmaceutical agents, the improvement comprising a timer for controlling the duration of the administration of the pharmaceutical agent.

### BRIEF DESCRIPTION OF THE DRAWINGS

FIG. 1 is a top perspective view of a nebulizer comprising the timer of the present invention.

FIG. 2 is close up of the timer dial of the present invention mounted on a nebulizer.

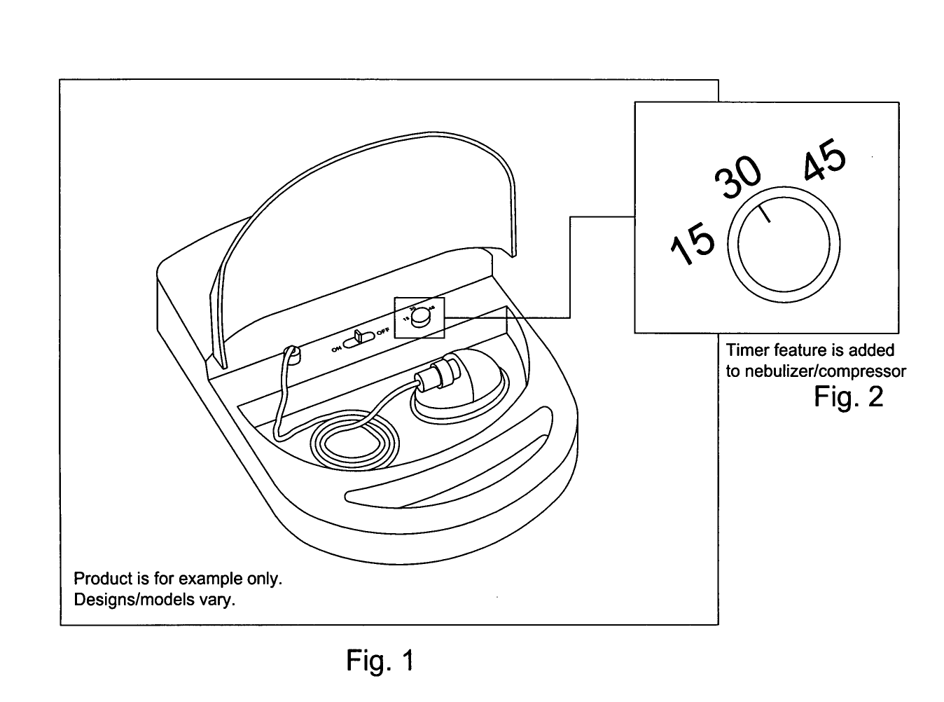

### DETAILED DESCRIPTION OF THE INVENTION

A nebulizer compressor is a device used to administer medication to people in forms of a liquid mist to the airways. Portable, electrically powered machines, nebulizers are commonly used in treating various respiratory diseases such as cystic fibrosis or asthma. Nebulizer compressors function by pumping air or oxygen through a liquid medicine, turning it into a vapor which is then inhaled by the patient. Typically, this vaporized medicine is inhaled through a tube-like mouthpiece, similar to those utilized in standard inhalers. These mouthpieces also serve to allow surrounding air to mix with the medicine, decreasing the unpleasantness of the vapor.

The inhaling apparatus, however, is sometimes replaced with a standard rubber face mask, similar to that used for inhaled anesthesia, particularly when treating young children or the elderly.

Depending on the health and medical condition of the patient, a typical nebulizer treatment will last between 15-20 minutes. It is not uncommon however, for the machine to run several minutes after the treatment has ended, simply because a respiratory therapist or other medical professional is caring for other patients and is simply too busy to turn the machine off.

Unfortunately, should a nebulizer run for even a few minutes after the liquid medicine has run out, the result can be that the machine overheats. Damaging the delicate components of the machine, this overheating can compromise the operational function of the unit, preventing accurate and measured delivery of medication with each following use. In worse case scenarios, an overheated nebulizer may catch fire, compromising the safety of the patient, as well as medical personnel.

Having recognized the potential for an improvement to the design of nebulizers which would prevent these machines from overheating, the present invention, hereinafter referred to as the Timer For Nebulizer Compressor, was developed. As the name implies, the Timer For Nebulizer Compressor is just that; an enhancement to the design of nebulizers which incorporates a simple timer and automatic shut off valve into its design.

A basic overview of this product is presented herein. The Timer For Nebulizer Compressor boasts the same basic design features of standard nebulizers. As such, this unique nebulizer boasts a shatterproof plastic casing which houses the unit motor and a piston driven or comparable compressor. Associated accessories such as a mouthpiece, medicine cup and elongated, flexible tubing are also included. The Timer For Nebulizer Compressor runs on standard AC power and can be connected to any power outlet. The novel feature of the Timer For Nebulizer Compressor however, is found in the previously mentioned timer. This timer is directly connected to the unit's shut off valve. As such, a therapist simply prepares the unit for use, at which point the timer is set for the time period required to administer the designated course of treatment. When the treatment was complete and the allotted time had passed, the timer is activated, automatically shutting the nebulizer off and thus preventing the unit from overheating.

The Timer For Nebulizer Compressor is an innovative invention which offers medical professionals and their patients a number of significant benefits and advantages. Foremost, the Timer For Nebulizer Compressor provides a practical enhancement to the design of nebulizer compressors which prevents these devices from overheating during use.

A specially designed nebulizer compressor boasting an integrated timer and automatic shut off valve, the Timer For Nebulizer Compressor immediately shuts down once the course of treatment was complete, thus ensuring the operational integrity of the machine. Preventing the damage to the motor and compressor which can occur if the unit overheats, the Timer For Nebulizer Compressor ensures that the machine runs smoothly and without interruption with each use. Most importantly, because the automatic, timed shut off valve prevents the unit from overheating, use of this product eliminates the fire hazards germane with nebulizer machines. Protecting the health and well being of the patient and medical personnel, the Timer For Nebulizer Compressor will prove an invaluable safety device and practical alternative to traditional nebulizer units. Durably constructed, the Timer For Nebulizer Compressor will withstand repeated, use with ease.

The Timer For Nebulizer Compressor is an innovative invention which provides medical professionals and their patients a practical enhancement to the design of nebulizers. A fully functioning nebulizer compressor boasting an automatic shut off valve, this unique nebulizer will never overheat, thus protecting the integrity of the machine and the safety of the patient and health care providers. Affordably priced, the Timer For Nebulizer Compressor will be well received by medical professionals and their patients.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. An improved nebulizer comprising a timer, comprising a shatterproof plastic casing which houses a unit motor and a piston driven or comparable compressor, further comprising a mouthpiece, medicine cup and elongated, and flexible tubing; wherein the nebulizer runs on standard AC power and is capable of being connected to any power outlet, and further comprising a timer, wherein the timer is directly connected to the unit's shut off valve.

2. A method for preventing the overheating of a nebulizer compressor comprising timing the duration of treatment of a nebulizer further comprising: a therapist preparing the unit for use, setting the timer for the time period required to administer the designated course of treatment, when the treatment is complete and the allotted time has passed, the timer is activated, automatically shutting the nebulizer off and thus preventing the unit from overheating.
